(19) United States
(12) Patent Application Publication
(10) Pub. No.: US 2020/0271621 A1
(43) Pub. Date: Aug. 27, 2020

(54) GAS SENSOR FOR DETECTING A TARGET GAS IN AN ENVIRONMENT

(71) Applicant: Sumitomo Chemical Company Limited, Tokyo (JP)

(72) Inventors: Nicholas Dartnell, Eltisley (GB); Simon Goddard, Impington (GB); Christopher Newsome, St. Ives (GB); Daniel Tobjork, Cambridgeshire (GB); Nir Yaacobi-Gross, Cambridgeshire (GB)

(73) Assignee: Sumitomo Chemical Company Limited, Tokyo (JP)

(21) Appl. No.: 16/651,283

(22) PCT Filed: Sep. 24, 2018
(86) PCT No.: PCT/EP2018/075830

§ 371 Date: Mar. 26, 2020

(30) Foreign Application Priority Data
Sep. 29, 2017 (GB) 1715847.8
Nov. 21, 2017 (GB) 1719329.3

(51) Int. Cl. G01N 27/414; G01N 33/00; G01N 27/12

(52) U.S. Cl. CPC G01N 27/4141; G01N 27/126; G01N 33/0047

(57) ABSTRACT

A gas sensor system is made up of a first gas sensor that is sensitive to both a target gas (200) and a secondary gas and a second sensor (300) that is only sensitive to the target gas. The response of the two gas sensors is processed to detect a presence of or a concentration of the target gas. The first sensor includes a semiconductor material that is sensitive to the presence of both the target and the secondary gas and electrodes that are sensitive to the presence of the target gas. The second sensor includes a semiconductor material that is sensitive to the presence of both the target and the secondary gas, but also includes a blocking layer on a surface of at least one of the electrodes that prevents the second gas interacting with the electrodes.

### BACKGROUND

Embodiments of the present application relate to semiconductor device gas sensors and the use of such sensors for the detection of gases, such as alkenes.

The use of thin film transistors as sensors is disclosed in, for example, Feng et al., “Unencapsulated Air-stable Organic Field Effect Transistor by All Solution Processes for Low Power Vapor Sensing” SCIENTIFIC REPORTS 6:20671 DOI: 10.1038/srep20671 and Besar et al., “Printable Ammonia Sensor Based on Organic Field Effect Transistor”, ORGANIC ELECTRONICS, Volume 15, Issue 11, pages 3221-3230 (November 2014).

Ethylene produced by plants can accelerate ripening of climateric fruit, the opening of flowers, and the shedding of plant leaves. 1-methylcyclopropene (1-MCP) is known for use in inhibiting such processes.

Robin et al., ORGANIC ELECTRONICS, Vol. 39, p 214-221 (2016) “Improvement of n-type OTFT Electrical Stability by Gold Electrode Modification” discloses modification of gold source and drain electrodes of an n-type OTFT using thiolated molecules.

### SUMMARY

Embodiments of the present disclosure provide a gas sensor capable of detecting gases, such as alkenes, and more particularly a gas sensor system capable of distinguishing between different gases, such as different alkenes.

In some embodiments of the present disclosure, a gas sensor is provided comprising a pair of electrodes and a semiconductor layer in electrical contact with both of the electrodes. The gas sensor includes a blocking layer disposed between a surface of at least one of the pair of electrodes and the semiconducting layer. The blocking layer is configured to block a particular gas from interacting with/causing a response from the gas sensor. In some embodiments, the gas sensor is used to measure/detect a second gas in the presence of the particular gas. Merely by way of example, a blocking layer of thiol may block the gas sensor from responding to 1-MCP, but may have no effect on the response of the sensor to ethylene. In this way, the gas sensor may be able to detect/measure ethylene in an atmosphere containing 1-MCP.

In a first aspect, according to some embodiments of the present disclosure, there is provided a gas sensor system comprising a first gas sensor and a second gas sensor. Each of the two gas sensors comprises a pair of electrodes and a semiconductor layer in electrical contact with both of the electrodes. The first gas sensor includes a blocking layer disposed between a surface of at least one of the pair of electrodes and the semiconducting layer. However, the second sensor does not include such a blocking layer. The blocking layer blocks the response of the first gas sensor to presence of a particular gas to which the second sensor, without the blocking layer, is responsive.

In a second aspect, according to some embodiments of the present disclosure, there is provided a method of identifying the presence and/or concentration of at least one target gas in an environment. The method comprises measuring a response of the first gas sensor, according to the first aspect, and measuring a response of the second gas sensor, according to the first aspect, and determining from the measured responses if the at least one target gas is present and/or determining a concentration of the at least one target gas.

### DESCRIPTION OF THE DRAWINGS

The invention will now be described in detail with reference to the Figures in which:

FIG. 1 illustrates a gas sensor system, according to some embodiments of the present disclosure;

FIG. 2 illustrates a bottom gate, bottom contact organic thin film transistor for use as a gas sensor, according to some embodiments of the present disclosure;

FIG. 3 illustrates a bottom gate, top contact organic thin film transistor for use as a gas sensor, according to some embodiments of the present disclosure;

FIG. 4 illustrates a top gate organic thin film transistor for use as a gas sensor, according to some embodiments of the present disclosure;

FIG. 5A illustrates a bottom contact horizontal chemiresistor for use as a gas sensor, according to some embodiments of the present disclosure;

FIG. 5B illustrates a top contact horizontal chemiresistor for use as a gas sensor, according to some embodiments of the present disclosure;

FIG. 6A illustrates a vertical chemiresistor for use as a gas sensor, according to some embodiments of the present disclosure, in which a bottom electrode has a blocking layer on a surface thereof;

FIG. 6B illustrates a vertical chemiresistor for use as a gas sensor, according to some embodiments of the present disclosure, in which a top electrode has a blocking layer on a surface thereof;

FIG. 7 is a graph of drain current versus time upon exposure to 1-MCP of a bottom gate OTFT having no blocking layer on the source and drain electrodes;

FIG. 8 is a graph of percentage change in drain current versus 1-MCP concentration for a bottom gate OTFT having no blocking layer on the source and drain electrodes;

FIG. 9 is a graph of drain current versus time upon exposure to 1-MCP of a bottom gate OTFT having a blocking layer on the source and drain electrodes;

FIG. 10 is a graph of drain current versus time for bottom gate OTFTs of a gas sensor according to an embodiment of the invention upon exposure to 1-MCP and ethylene.

FIG. 11A is a graph of current vs. bias voltage for a vertical chemiresistor having a blocking layer before and after exposure to 1-MCP;

FIG. 11B is a graph of current vs. bias voltage for a vertical chemiresistor without a blocking layer before and after exposure to 1-MCP;

FIG. 12A is a graph of percentage change in current vs. bias voltage for a vertical chemiresistor having a blocking layer before and after exposure to 1-MCP; and

FIG. 12B is a graph of percentage change in current vs. bias voltage for a vertical chemiresistor without a blocking layer before and after exposure to 1-MCP.

### DESCRIPTION

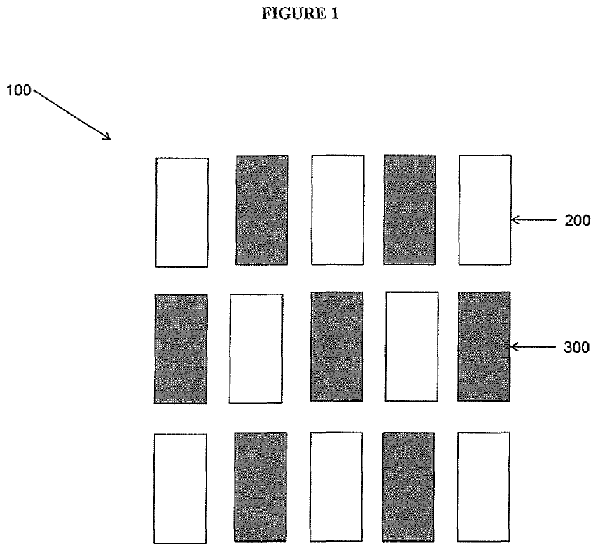

FIG. 1 is a schematic illustration of a gas sensor system 100 according to some embodiments of the present disclosure. The gas sensor system comprises a plurality of first sensors 200 and a plurality of second sensors 300. Each of the first and second sensors is capable of sensing one or more gases.

In the embodiment of FIG. 1, the gas sensor system comprises a plurality of each of the first and second sensors arranged in an array of alternating first and second sensors, however it will be appreciated that the first and second sensors may be provided in different first: second sensor ratios and/or in different configurations relative to one another. In some embodiments, the gas sensor system may comprise only one first sensor and/or only one second sensor. The gas sensor system may comprise one or more further gas sensors, optionally one or more OTFT gas sensors, which are exposed to the atmosphere when the gas sensor system is in use.

In an embodiment, the first and second sensors are organic thin film transistors (OTFTs) and may be bottom-gate organic thin film transistors (BG-OTFTs) or top gate organic thin film transistors.

Each BG-OTFT may be a bottom contact or top contact device.

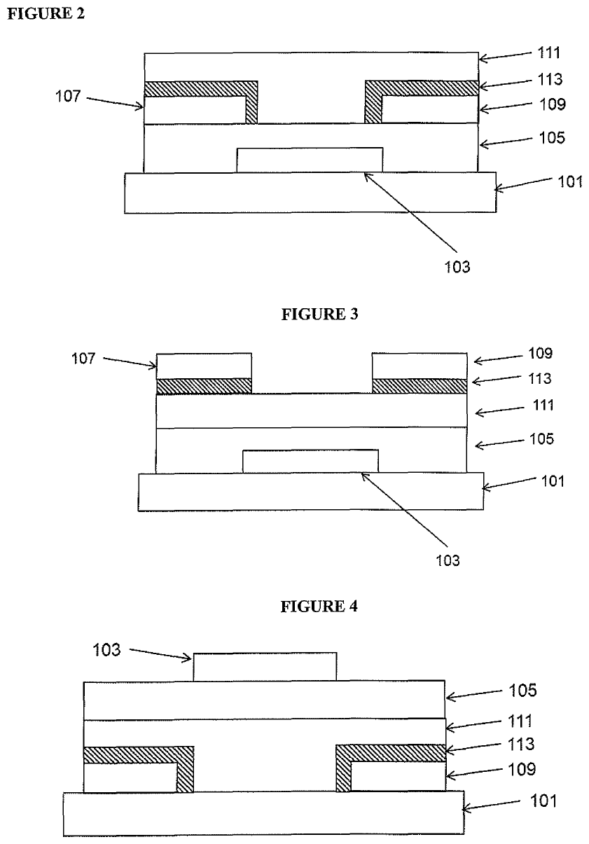

FIG. 2 is a schematic illustration of a bottom contact BG-OTFT suitable for use as a first BG-OTFT gas sensor in a gas sensor system as described herein. The bottom contact BG-OTFT comprises a gate electrode 103 over a substrate 101; source and drain electrodes 107, 109; a blocking layer 113 on a surface of the source and drain electrodes; a dielectric layer 105 between the gate electrode and the source and drain electrodes; and an organic semiconductor layer 111 contacting blocking layer. The organic semiconductor layer 111 may at least partially or completely cover the source and drain electrodes.

As used herein, by a material “over” a layer is meant that the material is in direct contact with the layer or is spaced apart therefrom by one or more intervening layers.

As used herein, by a material “on” a layer is meant that the material is in direct contact with that layer.

A layer “between” two other layers as described herein may be in direct contact with each of the two layers it is between or may be spaced apart from one or both of the two other layers by one or more intervening layers.

FIG. 3 is a schematic illustration of a top-contact BG-OTFT suitable for use as a first BG-OTFT gas sensor in a gas sensor system as described herein. The top-contact BG-OTFT is as described with reference to FIG. 2 except that the organic semiconductor layer 111 is between the dielectric layer 105 and the source and drain electrodes 107, 109.

FIG. 4 is a schematic illustration of a top gate OTFT suitable for use as a first OTFT gas sensor in a gas sensor system as described herein. The top gate OTFT comprises source and drain electrodes 107, 109; a blocking layer 113 on a surface of the source and drain electrodes; an organic semiconductor layer 111 contacting blocking layer 113; and a dielectric layer 105 between the gate electrode 103 and the organic semiconductor layer. The dielectric layer of the top-gate OTFT is a gas-permeable material, preferably an organic material, which allows permeation of the gas or gases to be sensed through the dielectric layer to the organic semiconducting layer. First and second OTFT sensors as described herein are preferably BG-OTFT sensors, more preferably bottom contact BG-OTFTs.

The organic semiconducting layer of the first OTFT is preferably in direct contact with the blocking layer, for example as illustrated in FIGS. 2-4. In other embodiments, the blocking layer may be spaced apart from the organic semiconducting layer by an organic charge-transporting layer in direct contact with the blocking layer. An organic charge-transporting layer as described herein may be as disclosed in WO 2016/001095, the contents of which are incorporated herein by reference.

The blocking layer at least partially covers a surface of the first and second electrodes, such as the source and drain electrodes in the case where the first and second sensors are OTFTs, which would, in the absence of the blocking layer, be in direct contact with the organic semiconducting layer or, if present, the charge-transporting layer. The blocking layer on the first and second electrodes is in direct contact with the organic semiconducting layer or, if present, the organic charge-transporting layer, thereby forming an interface between the first and second electrodes and the organic layer. Blocking layer 113 of the first OTFT may partially or completely prevent an atmospheric gas from binding to the surface of the first and second electrodes on which the blocking layer has been formed.

With reference to FIG. 3, it may be possible for an atmospheric gas to bind to an outer surface of the first and second electrodes, if they are not covered with a blocking layer. However, this outer surface is not at an interface with the organic semiconducting layer and so a gas binding to this surface may have little or no effect on charge injection into the organic semiconducting layer.

In embodiments of the present disclosure, the second sensor does not have blocking layer 113 and so an atmospheric gas coming into contact with the first and second electrodes at the electrode/semiconductor layer interface is not blocked by a blocking layer from binding to the surface of the first and second electrodes.

The first and second gas sensors may be first and second chemiresistors. Chemiresistors as described herein may be vertical or horizontal chemiresistors.

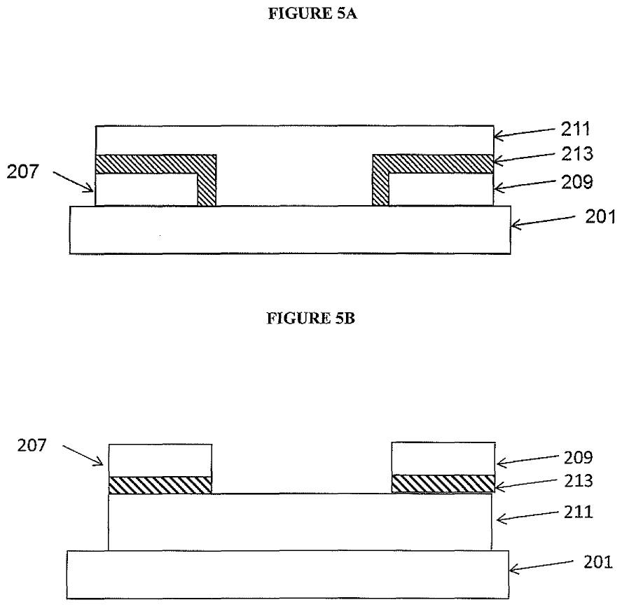

FIG. 5A illustrates a bottom contact horizontal chemiresistor according to an embodiment suitable for use as a first sensor as described herein. By “bottom contact chemiresistor” as used herein is meant that electrodes of the chemiresistor lie between a substrate and an organic semiconducting layer of the chemiresistor

The chemiresistor comprises first and second electrodes 207 and 209 having a blocking layer 213 formed thereon. An organic semiconductor layer 211 is provided between, and in electrical connection with, the first and second electrodes. The first and second electrodes may be interdigitated. The chemiresistor may be supported on any suitable substrate 201, for example a glass or plastic substrate.

FIG. 5B illustrates a top contact horizontal chemiresistor according to an embodiment suitable for use as a first sensor as described herein. By “top contact chemiresistor” as used herein is meant that an organic semiconducting layer of the chemiresistor lies between electrodes and a substrate of the chemiresistor

Integers of the chemiresistor of FIG. 5B are as described with reference to FIG. 5A. Blocking layer 213 lies between the organic semiconducting layer and the electrodes.

The first and second electrodes of a horizontal chemiresistor as described herein may be separated by a distance of between 5-500 microns, optionally 50-500 microns.

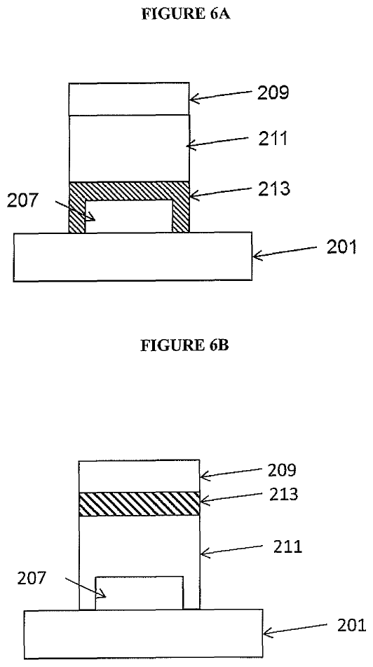

In another embodiment (not shown) a first sensor is a horizontal chemiresistor as described with reference to FIGS. 5A or 5B except that only one of the first and second electrodes have a blocking layer foamed thereon, the other of the first and second electrodes being in direct contact with the semiconductor layer. FIG. 6A illustrates a vertical chemiresistor according to an embodiment suitable for use as a first sensor as described herein. The chemiresistor comprises a first, bottom electrode 207 having a blocking layer 213 formed thereon; a second, top electrode 209 over the first electrode; and an organic semiconductor layer 211 between, and in electrical connection with, the first and second electrodes. The bottom electrode 207 lies between the substrate and both the organic semiconducting layer 211 and the second, top electrode 209.

FIG. 6B illustrates another vertical chemiresistor according to an embodiment suitable for use as a first sensor as described herein. Integers of the chemiresistor of FIG. 6B are as described with reference to FIG. 6A. Blocking layer 213 lies between the organic semiconducting layer and the second, top electrode 209.

In a further embodiment, each of the first and second electrodes of a vertical chemiresistor have a blocking layer between the electrode and the semiconducting layer.

The first and second electrodes of a vertical chemiresistor as described herein may be separated by a distance of between 20 nm-10 microns, optionally 50-500 nm.

A gas contacting an electrode surface, such as a gas having a dipole moment, may result in a change in work function at the electrode surface, for example as a result of binding of the gas to the electrode surface. Schottky current dependence on work function may mean that even a relatively small change in work function Δϕ has a large effect on currents J₁ and J₂ at these work functions:

J₂/J₁=e^{-(Δϕ/kT)}

An electrode having a blocking layer thereon may undergo a smaller change in work function upon exposure to a gas than the same electrode without a blocking layer thereon. Optionally, the work function of an electrode having a blocking layer thereon does not change upon exposure to a gas.

Use of first and second gas sensors with and without blocking layers as described herein may provide improved identification of a gas in an atmosphere and/or improved differentiation between different gases in an atmosphere, such as an atmosphere containing a gas with a dipole moment, such as 1-MCP and a gas without a dipole moment, such as ethylene.

The current of devices as described herein at a given voltage is suitably limited by the electrode-semiconductor contact resistance. The presence of a blocking layer on an electrode as described herein may limit an effect that a gas may have on the contact resistance between the electrode and the semiconducting layer.

The first and second sensors may differ only in that the second sensor does not have a blocking layer or there may be one or more further differences between the first and second sensors. The first and second sensors may be produced simultaneously, the production differing at least, optionally only, in that the first sensor is provided with a blocking layer as described herein whereas the second sensor is not.

In use, the organic semiconducting layers of each of the first and second sensors and, if present, any of the further gas sensors of the gas sensor system, are exposed to a gaseous atmosphere and the response of the first and second sensors to the atmosphere resulting from absorption of one or more gases in the atmosphere may be measured.

In some embodiments, the gas sensor system is a gas sensor system for sensing an alkene, 1-methylcyclopropene (1-MCP) and/or ethylene. In some embodiments, the gas sensor system is used for detecting ethylene and/or 1-MCP in an environment in which one or both of ethylene and 1-MCP may be present.

The present inventors have found that the responses of first and second sensors to 1-MCP are significantly different. This difference in response may be used to determine whether a gas detected by the gas sensor system is 1-MCP or another gas, in particular ethylene, present in the environment.

In some embodiments, a processor, software, a computer and/or the like is in communication with the first and the second gas sensors of the gas sensor system. Because one of the first and the second sensors includes a blocking layer and the other sensor does not, the first and the second sensor respond differently to the presence of two target gases, such as, for example ethylene and 1-MCP. As a result, in some embodiments of the present disclsoure, the processor may take the outputs from the first and the second sensor and may detect the presence and or concentration of one or both of the target gases.

In use, the gas sensor system may be placed in an environment in which alkenes may be present in the environmental atmosphere, for example a warehouse in which harvested climateric fruits and/or cut flowers are stored and in which ethylene may be generated.

The presence and/or concentration of ethylene may be determined using the gas sensor system. If ethylene concentration reaches or exceeds a predetermined threshold value, which may be any value greater than 0, then 1-MCP may be released from a 1-MCP source to retard the effect of the ethylene, such as ripening of fruit or opening of flowers in the environment.

In some embodiments, 1-MCP may be released into the atmosphere, if the 1-MCP concentration falls to or below a threshold 1-MCP concentration value as determined by the gas sensor system. The threshold 1-MCP concentration value may be 0 or a positive value.

1-MCP may be released automatically from a 1-MCP source or an alert or instruction may be generated to manually release 1-MCP from a 1-MCP source in response to signal from the gas sensor system upon determination that 1-MCP concentration is at or below a threshold that is a positive value and/or in response to a determination that ethylene concentration is at or exceeds a threshold which may be 0 or a positive value.

The gas sensor may be in wired or wireless communication with a controller which controls automatic release of 1-MCP from a 1 -MCP source and /or a user interface providing information on the presence and/or concentration of ethylene and /or 1-MCP in the environment.

An environment in which an alkene may be present may be divided into a plurality of regions, if the concentration of an alkene or alkenes may differ between regions. In some embodiments, each region may comprising a gas sensor system, according to embodiments of the present invention and a source of 1-MCP. For example, a warehouse may comprise a plurality of regions and a plurality of gas sensors, in accordance with the present disclosure, may be used to monitor one or more gases in the separate regions.

The gas sensor system may comprise one or more control gas sensors, for example one or more OTFT gas sensors, to provide a baseline for measurements made by the first and second sensors to take into account variables, such as one or more of humidity, temperature, pressure, variation of sensor parameter measurements over time (such as variation of OTFT sensor drain current over time), and gases other than a target gas or target gases in the atmosphere. One or more control gas sensors may be isolated from the atmosphere, for example by encapsulation of one or more of the control sensor, to provide a baseline measurement other than gases in the atmosphere.

The response of the first and second sensors of the gas sensor system to background gases other than the target gases for detection, for example air or water vapour, may be measured prior to use to allow subtraction of the background from measurements of the gas sensor system when in use.

Each of the sensors of the gas sensor system may be supported on a common substrate and/or contained in a common housing. In use, each sensor may be connected to a common power source, or two or more of the sensors may be powered by different power sources. In use, power to all of the sensors of the gas sensor may be controlled by a single switch or power to two or more of the sensors may be controlled by different switches.

Blocking Layer

In some embodiments, the blocking layer of the first sensor is a monolayer formed on a surface of the first and second electrodes. A blocking layer may, in some embodiments, be formed from a binding compound of formula (I):

R—X

where, R is an organic residue and X is a binding group for binding to the surface of the source and drain electrodes. The binding group X may bind to the source and drain electrodes to form a self-assembled monolayer.

X may be selected according to the material of the source and drain electrodes. In some embodiments, X is a thiol or a silane group. A thiol group X may, in some embodiments, be used with source and drain electrodes comprising gold.

In some embodiments, R is a C₁₋₃₀ hydrocarbyl group which may be unsubstituted or substituted with one or more substituents. Exemplary C₁₋₃₀ hydrocarbyl groups are: C₆₋₂₀ aromatic groups, preferably phenyl, phenyl with one or more C₁₋₂₀ alkyl groups; and phenyl-C₁₋₂₀ alkyl which may be substituted with one or more C₁₋₂₀ alkyl groups.

In some embodiments, a substituent of the C₁₋₃₀ hydrocarbyl group is fluorine, and one or more H atoms of the C₁₋₃₀ hydrocarbyl group may be replaced with fluorine.

Exemplary compounds of formula (I) are:

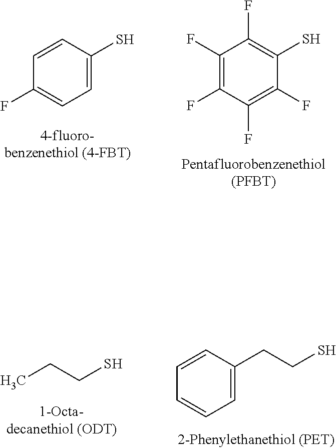

The blocking layer may alter the work function of the electrode or electrodes it is formed on. The blocking layer may be selected according to the effect, if any, of the blocking layer on the work function of the first and/or second electrodes and the required charge injection requirements of the first sensor such as the work function—organic semiconductor highest occupied molecular orbital (HOMO) gap in the case of a p-type BG-OTFT or the work function—organic semiconductor lowest unoccupied molecular orbital (LUMO) gap in the case of a n-type BG-OTFT.

In some embodiments, the work function of the source and drain electrodes of a p-type OTFT is increased following treatment. In some embodiments, the work function may be increased to a value of 5.0 or more. The HOMO of a p-type semiconducting material may, in some embodiments, be at least 5.0 eV or in the range of about 5.0-5.5 eV.

A monolayer may be formed on the first electrode, or on the first and second electrodes, by depositing the binding compound on the electrode or electrodes, for example from a solution of the binding compound in one or more solvents. The binding compound may be selectively deposited onto the first and second electrodes only, or may be deposited by a non-selective process such as spin-coating or dip-coating.

A bottom-contact BG-OTFT may be formed by depositing the binding compound onto the source and drain electrodes over a dielectric layer and then depositing the organic semiconducting layer. Binding compound which is not bound to the source and drain electrodes, for example binding compound on the dielectric layer following a non-selective deposition process, may be removed by washing.

The binding compound may be formed over first and/or second electrodes of a horizontal chemiresistor. The binding compound may be formed over a first, bottom electrode only of a vertical chemiresistor. The blocking layer may be a material which is absorbed onto the surface of an electrode or to electrodes of the first sensor.

Electrodes

In some embodiments, the first and second electrodes of the first and second sensors comprise source and drain electrodes of first and second OTFTs, or first and second electrodes of first and second chemiresistors. The first and second electrodes may be selected from a wide range of conducting materials for example a metal (e.g. gold), metal alloy, metal compound (e.g. indium tin oxide) or conductive polymer. The first and second electrodes of the first sensor may be selected according to the material of the blocking layer.

In the case of an OTFT, the gate electrode may be selected from any conducting material, for example a metal (e.g. aluminium), a metal alloy, a conductive metal compound (e.g. a conductive metal oxide such as indium tin oxide) or a conductive polymer.

The length of the channel defined between the source and drain electrodes of the first and second source and drain and gate electrodes of the first and second OTFTs may be up to 500 microns, but preferably the length is less than 200 microns, more preferably less than 100 microns.

Semiconductor Layer

The invention has been described with reference to sensors comprising organic semiconductors, however it will be appreciated that an inorganic semiconductor may be used in place of an organic semiconductor as described anywhere herein.

Organic semiconductors as described herein may be selected from conjugated non-polymeric semiconductors; polymers comprising conjugated groups in a main chain or in a side group thereof; and carbon semiconductors such as graphene and carbon nanotubes.

An organic semiconductor layer of the first or second sensors may comprise or consist of a semiconducting polymer and/or a non-polymeric organic semiconductor. The organic semiconductor layer may comprise a blend of a non-polymeric organic semiconductor and a polymer. Exemplary organic semiconductors are disclosed in WO 2016/001095, the contents of which are incorporated herein by reference.

The organic semiconductor layer of the first and second BG-OTFTs may comprise or consist of only one organic semiconductor. The organic semiconductor layer of first and second top-gate organic thin film transistors may be a mixture of a non-polymeric and polymeric organic semiconductor.

The organic semiconducting layer may be deposited by any suitable technique, including evaporation and deposition from a solution comprising or consisting of one or more organic semiconducting materials and at least one solvent. Exemplary solvents include benzenes with one or more alkyl substituents, preferably one or more C₁₋₁₀ alkyl substituents, such as toluene and xylene; tetralin; and chloroform. Solution deposition techniques include coating and printing methods, for example spin coating dip-coating, slot-die coating, ink jet printing, gravure printing, flexographic printing and screen printing.

In some embodiments, the organic semiconducting layer of an organic thin film transistor has a thickness in the range of about 10-200 nm.

Exemplary inorganic semiconductors include, without limitation, n-doped silicon; p-doped silicon; compound semiconductors, for example III-V semiconductors such as GaAs or InGaAs; doped or undoped metal oxides; doped or undoped metal sulfides; doped or undoped metal selenides; or doped or undoped metal tellurides.

Dielectric Layer

In some embodiments, the dielectric layer of the first and second OTFTs comprises a dielectric material. In some embodiments, the dielectric constant, k, of the dielectric material is at least 2 or at least 3. The dielectric material may be organic, inorganic or a mixture thereof. In some embodiment, inorganic materials used may include SiO₂, SiNx and spin-on-glass (SOG). Preferred organic materials are polymers and include insulating polymers such as poly vinylalcohol (PVA), polyvinylpyrrolidine (PVP), acrylates such as polymethylmethacrylate (PMMA) and benzocyclobutanes (BCBs), poly(vinyl phenol) (PVPh), poly(vinyl cinnamate) P(VCn), poly(vinylidene fluoride-co-hexafluoropropylene) P(VDF-HFP), P(VDF-TrFE-CTFE), and self-assembled monolayers, e.g. silanes, on oxide. The polymer may be crosslinkable. The insulating layer may be formed from a blend of materials or comprise a multi-layered structure. In the case of a bottom-gate device, the gate electrode may be reacted, for example oxidised, to form a dielectric material.

The dielectric material may be deposited by thermal evaporation, vacuum processing or lamination techniques as are known in the art. Alternatively, the dielectric material may be deposited from solution using, for example, spin coating or ink jet printing techniques and other solution deposition techniques discussed above. In the case of a bottom gate OTFT, the dielectric material should not be dissolved if an organic semiconductor is deposited onto it from solution. In the case of a top-gate OTFT, the organic semiconductor layer should not be dissolved if the dielectric is deposited from solution.

Techniques to avoid such dissolution include: use of orthogonal solvents for example use of a solvent for deposition of the organic semiconducting layer that does not dissolve the dielectric layer in the case of a bottom gate device or vice versa in the case of a top gate device; cross linking of the dielectric layer before deposition of the organic semiconductor layer in the case of a bottom gate device; or deposition from solution of a blend of the dielectric material and the organic semiconductor followed by vertical phase separation as disclosed in, for example, L. Qiu, et al., Adv. Mater. 2008, 20, 1141.

In some embodiments, the thickness of the dielectric layer may be in the range less than about 2 micrometres, or less than about 500 nm. The substrate of a sensor as described herein may be any insulating substrate, e.g., glass, plastic and/or the like.

Use of first and second sensors has been described herein with reference to 1-MCP and ethylene, however it will be appreciated that first and second sensors and gas sensor systems comprising the sensors described herein may be used in detection of strained alkenes, for example, alkenes comprising a cyclopropene or cyclobutene group, of which alkylpropenes such as 1-MCP are examples; in detection of aliphatic alkenes, optionally ethylene, propene, 1-butene or 2-butene; and/or in detection of compounds with a dipole moment, such as hydrocarbons which do not have a mirror plane bisecting a carbon-carbon bond of the hydrocarbon. Preferably, compounds with a dipole moment as described herein have a dipole moment of greater than 0.2 Debyes optionally greater than 0.3 or 0.4 Debyes.

Blocking Layer Effect on Work Function

The effects on gold work function of: (i) exposure to 1-MCP, (ii) formation of blocking layers, and (iii) formation of blocking layers followed by exposure to 1-MCP were studied.

Following formation of gold contacts on a glass substrate, the blocking layer was formed by immersing the substrate in a solution of the blocking material in isopropyl alcohol (0.14 μL/ml) for a period of 2 minutes. The solution was then removed by spinning the substrate and rinsing it with IPA to remove excess thiol and the substrate was dried at 60° C. for 10 minutes.

With reference to Table 1, exposure to 1-MCP of gold without a blocking layer results in a much larger change in work function than exposure to 1-MCP of gold with a blocking layer. Without wishing to be bound by any theory, the smaller change in work function is when a blocking layer is present is due to blocking of 1-MCP from binding to gold.

Work functions given in Table 1 are as measured by AC2 photoelectron spectrometry.

TABLE 1

| | Wf before 1-MCP | Wf after 1-MCP | |
|---|---|---|---|
| Material | exposure, eV | exposure, eV | Wf change, eV |
| Gold | −4.8 | −4.55 | +0.25 |
| Gold + 4-FBT | −5.05 | −5.10 | −0.05 |
| Gold + ODT | −4.60 | −4.70 | −0.1 |
| Gold + PFBT | −5.60 | −5.50 | +0.1 |
| Gold + PET | −4.55 | −4.65 | −0.1 |

First BG-OTFT

On a PEN substrate carrying an aluminium gate electrode was formed a crosslinked dielectric layer by spin-coating and crosslinking an insulating polymer to a thickness of 60-300 nm. Gold source and drain electrodes were formed on the dielectric layer by thermal evaporation. A monolayer of 4-fluorobenzenethiol was formed on the surface of the source and drain electrodes as described above. Semiconducting Polymer 1 was formed over the dielectric layer and source and drain electrodes by spin-coating to a thickness of 40 nm to form bottom contact BG-OTFT Device Example 1.

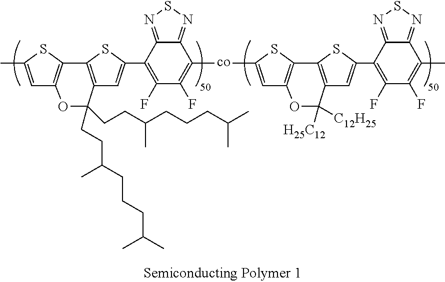

Second BG-OTFT

A second BG-OTFT was formed as described for the first BG-OTFT except that the gold source and drain electrodes were not treated with 4-fluorobenzenethiol.

1-MCP Response of BG-OTFTs

The response of the first and second BG-OTFTs to exposure to 1-MCP gas was measured by monitoring the level of the drain current as a function of time. The OTFT was driven at a constant finite voltage of Vg=Vds=−4V.

An alpha-cyclodextrin matrix containing 1-MCP (4.3 wt %) was added to water to displace the 1-MCP into a bottle purged with nitrogen (50 cc/min). The nitrogen gas carried the 1-MCP through a gas tight container containing the BG-OTFT.

The second OTFT was exposed to 1-MCP at concentrations of 50, 250 and 500 ppb. A recovery in drain current was observed in a pure nitrogen environment.

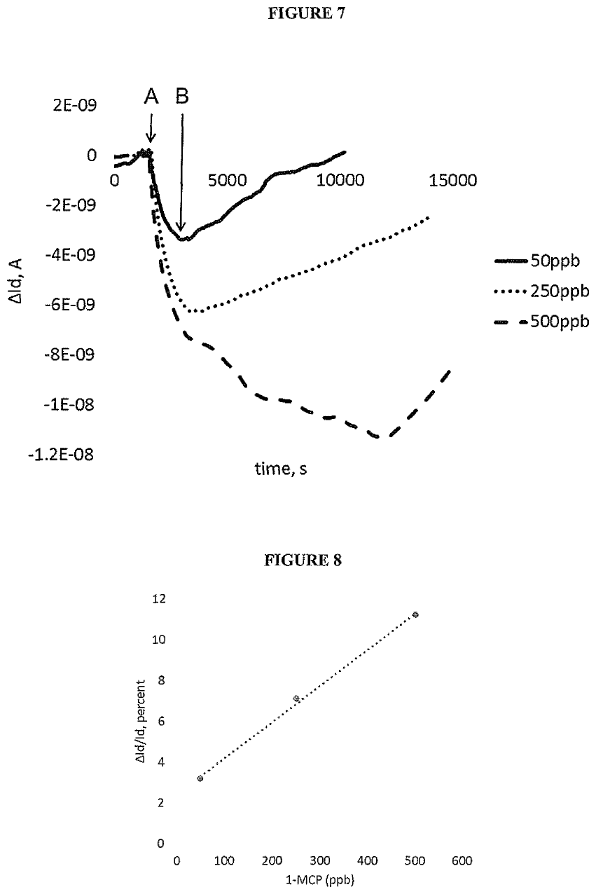

With reference to FIG. 7, the drain current of the second BG-OTFT, without treated source and drain electrodes, decreases upon exposure to an increasing concentration of 1-MCP at time point A and at least partially recovers upon displacement of 1-MCP in the environment with nitrogen gas starting at time point B.

With reference to FIG. 8, the percentage change in drain current is proportional to the 1-MCP concentration.

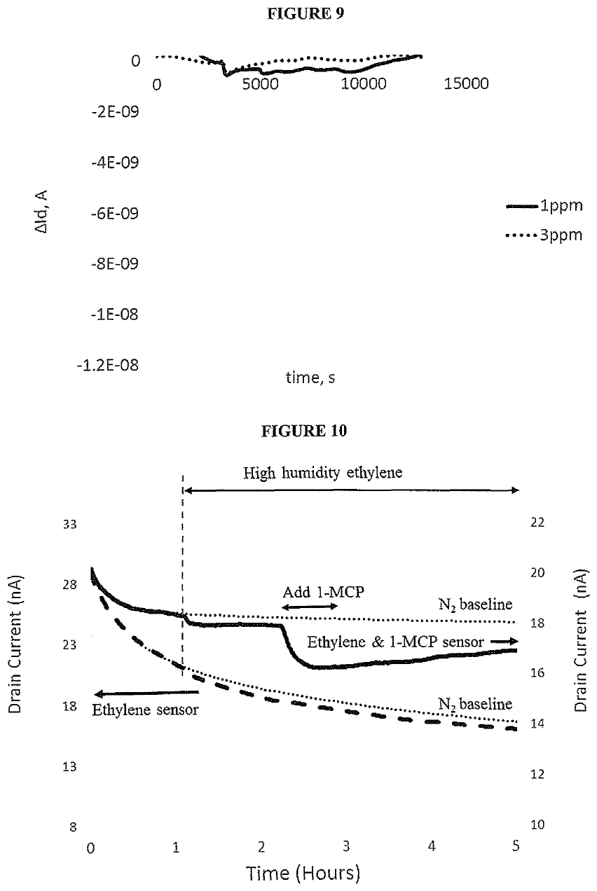

With reference to FIG. 9, the drain current of the first BG-OTFT, with treated source and drain electrodes, shows little change upon exposure to 1-MCP as described above with reference to FIG. 7, even at 1 ppm or 3 ppm concentration (i.e. 1-MCP concentrations 2 or 6 times greater than the highest concentration that the second BG-OTFT was exposed to).

Without wishing to be bound by any theory, it is believed that 1-MCP may bind to gold source and drain electrodes of the second BG-OTFT but little or no such binding is possible following formation of the blocking layer.

Sensor Example 1

First and second BG-OTFTs described above were both exposed to an atmosphere in which high humidity ethylene was firstly introduced followed by introduction of 1-MCP.

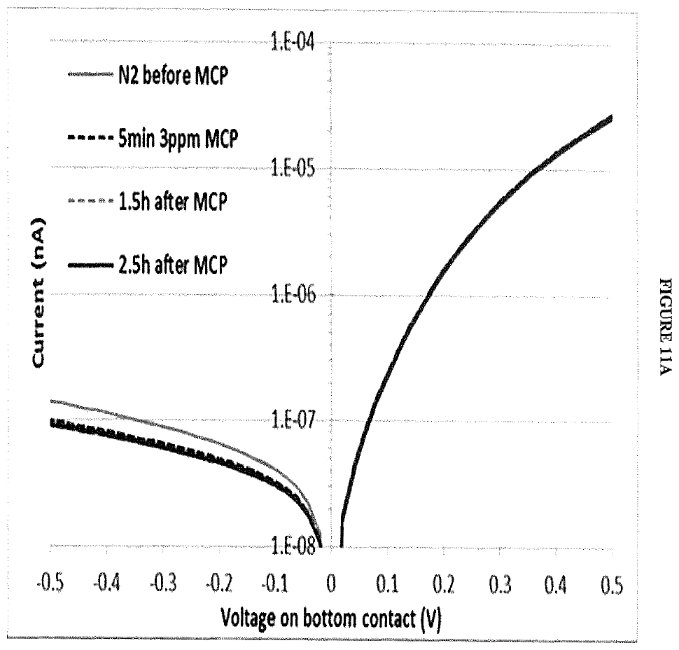

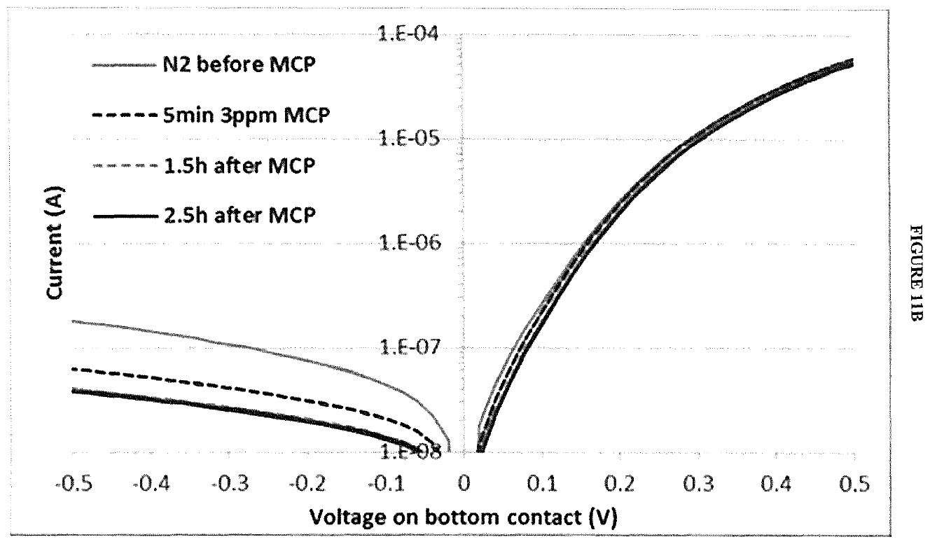

With reference to FIG. 10, the first OTFT sensor shows a fall in drain current upon introduction of ethylene but no response upon introduction of 1-MCP. In contrast, the second OTFT sensor shows a fall in drain current upon introduction of ethylene and upon introduction of 1-MCP. Consequently, 1-MCP concentration may be determined by subtracting a response of the first OTFT from the response of the second OTFT (if necessary, taking into account any calibration required due to any difference in response of the first and second OTFT sensors to ethylene gas and/or any measured response made by the first OTFT to 1-MCP).

Without wishing to be bound by any theory, ethylene may absorb into the organic semiconductor layer of the first and second OTFT sensors, thereby changing the drain current of both devices, whereas 1-MCP cannot absorb into the organic semiconductor layer, or cannot do so to the same extent as ethylene. Again, without wishing to be bound by any theory this may be due to the larger size of 1-MCP (4 carbon atoms vs. 2 carbon atoms of ethylene).

First Vertical Chemiresistor

A monolayer of 4-fluorobenzenethiol was formed on a first gold electrode supported on a glass substrate using the method described above. Semiconducting Polymer 1 was deposited onto the thiol-treated gold electrode by spin-coating to form a 300 nm thick semiconducting layer. A second gold electrode was formed on the semiconducting layer. The first and second electrodes were connected to apparatus for measuring the response of the chemiresistor on application of bias.

Second Vertical Chemiresistor

A second vertical chemiresistor was formed as described for the first vertical chemiresistor except that the first gold electrode was not treated with thiol.

1-MCP Response of Chemiresistors

The current of the first and second chemiresistors was measured upon application of a bias of between −0.5 to +0.5 V on the second electrode under the following conditions:
Nitrogen Atmosphere
0, 5, 10, 20, 90 and 150 minutes after introduction of 1-MCP.

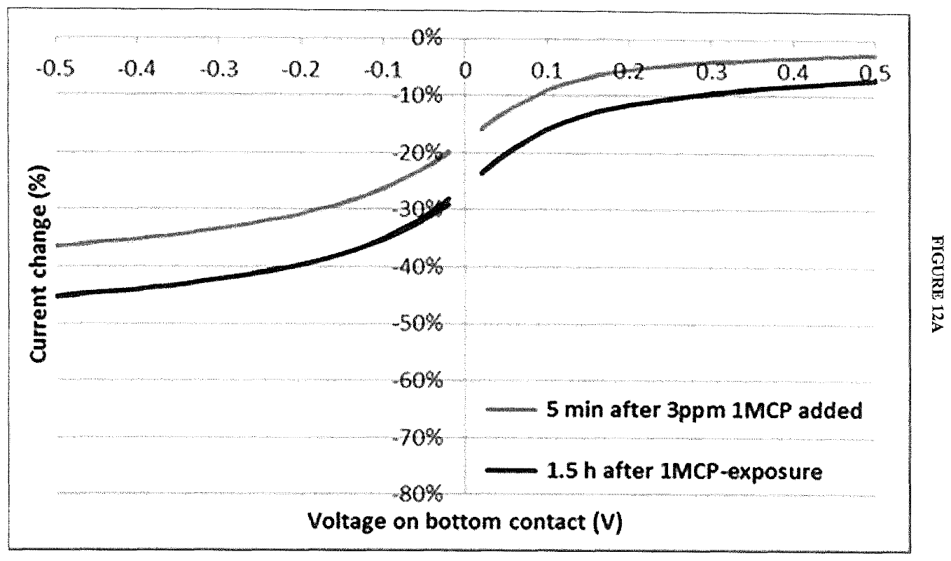

FIGS. 11A and 11B provide a comparison of the response of the first and second chemiresistor respectively to 1-MCP in terms of measured current as a function of voltage applied to the first electrode.

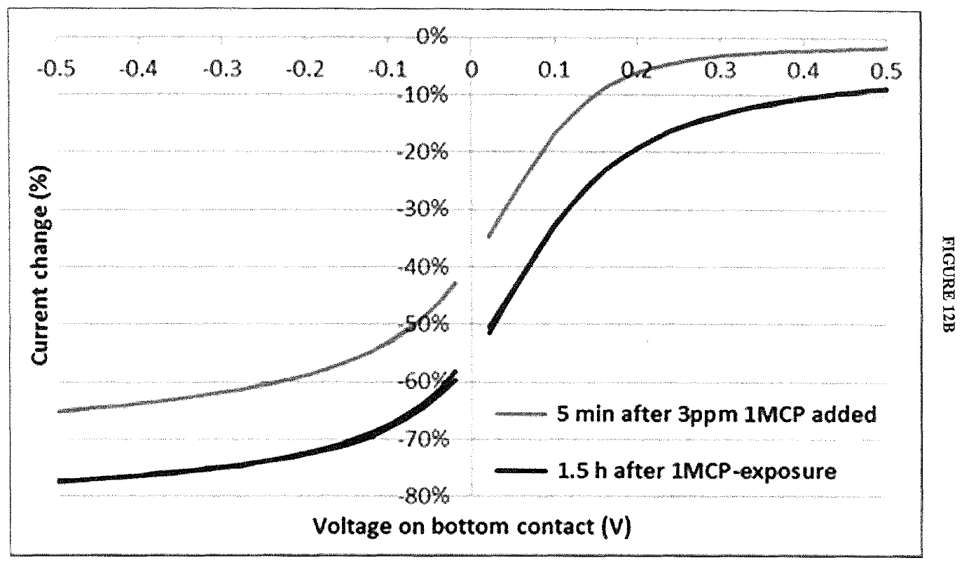

FIGS. 12A and 12B provide a comparison of the response of the first and second chemiresistor respectively to 1-MCP in terms of percentage change in current as a function of voltage applied to the first electrode.

As shown in these figures, the response to 1-MCP is significantly smaller for the first vertical chemiresistor, particularly at negative bias.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A gas sensor system for detecting a presence and/or a concentration of a target gas in an environment, comprising:
a first gas sensor comprising first and second electrodes and a semiconductor layer in electrical contact with the first and second electrodes;
a second gas sensor comprising first and second electrodes and a semiconductor layer in electrical contact with the first and second electrodes and a blocking layer on a surface of at least one of the first and second electrodes and disposed between the first electrode and/or the second electrode and the semiconducting layer; and
a processor configured to process the presence and/or the concentration of the target gas in the atmosphere from a first response from the first gas sensor and a second response from the second gas sensor.

2. A gas sensor system according to claim 1, wherein the first and second gas sensors comprise first and second thin film transistors, and wherein the first and second electrodes of the first and second gas sensors comprise source and drain electrodes of the thin film transistors.

3. A gas sensor system according to claim 2, wherein the first and second thin film transistors are bottom gate thin film transistors (BG-TFTs).

4. A gas sensor system according to claim 3 wherein the first and/or the second BG-TFT is a bottom contact TFT.

5. A gas sensor system according to claim 3 wherein the first and/or the second BG-TFT is a top contact TFT.

6. A gas sensor system according to claim 2, wherein the first and second thin film transistors comprise top gate thin film transistors (BG-TFTs).

7. A gas sensor system according to claim 2 wherein the TFTs comprise organic TFTs and the semiconducting layer of the TFTs comprises an organic semiconducting layer.

8. A gas sensor system according to claim 1, wherein the first and the second gas sensors comprise first and second chemiresistors.

9. A gas sensor system according to claim 8, wherein the first and second chemiresistors comprise vertical chemiresistors.

10. A gas sensor system according to claim 1, wherein the blocking layer comprises a monolayer on the surface of at least the first electrode of the first sensor.

11. A gas sensor system according to 10, wherein the blocking layer comprises a blocking compound comprising a thiol group.

12. A gas sensor system according to claim 11, wherein the first and second electrodes of the first and the second gas sensors comprise gold.

13. A gas sensor system according to claim 1, wherein the semiconductor layer of the first gas sensor is in direct contact with the blocking layer.

14. A gas sensor system according to claim 1, wherein the semiconductor layer of the second gas sensor is in direct contact with the first and second electrodes of the second gas sensor.

15. A gas sensor system according to claim 1, wherein the first response of the first gas sensor differs from the second response of the second gas sensor in a presence of 1-methylcyclopropene.

16. A gas sensor system according to claim 15, wherein the first and second gas sensors comprise first and second OTFTs and the response of the first OTFT differs from the response of the second OTFT in at least one of: amount of change in a drain current and rate of change in the drain current.

17. A method of determining a presence and/or a concentration of at least one target gas in an environment containing a secondary gas, the method comprising:
measuring a first response of a first gas sensor, wherein the first gas sensor comprises a TFT with first and second electrodes contacting a semiconductor material and the semiconductor material is configured to interact with both the target gas and the secondary gas and the first and the second electrodes are configured to interact with the target gas, and wherein the first response of the first gas sensor is produced by the interaction of the semiconductor material with the target gas and the secondary gas and the interaction of the first and the second electrodes with the target gas;
measuring a second response of a second gas sensor, wherein the second gas sensor comprises a TFT comprising first and second electrodes contacting a semiconductor material and a blocking layer disposed on at least one of the first and the second electrodes and configured to block an interaction between the target gas and the at least one of the first and second electrodes, and wherein the second response of the second gas sensor is produced by the interaction of the semiconductor material with the target gas and the secondary gas;
determining from the first and the second responses the presence and/or concentration of the target gas.

18. A method according to claim 17, wherein the target gas and/or the secondary gas comprises an alkene.

19. A method according to claim 17, wherein the target gas comprises one of ethylene and 1-methylcyclopropene.

20. A method according to claim 17, wherein the target secondary gas comprises one of ethylene and 1-methylcyclopropene.
